(19) United States
(12) Patent Application Publication
(10) Pub. No.: US 2019/0100731 A1
(43) Pub. Date: Apr. 4, 2019

(54) EX-VIVO INTESTINAL CULTURE MODEL, METHODS OF PRODUCING SAME AND USES THEREOF

(71) Applicants: The State of Israel, Ministry of Agriculture & Rural Development, Agricultural Research Organizat, Rishon-LeZion (IL); Mor Research Applications Ltd., Tel-Aviv (IL)

(72) Inventors: Hinanit KOLTAI, Rishon-LeZion (IL); Yoram KAPULNIK, Karmey Yosef (IL); Marcelo FRIDLENDER, Mazkeret Batia (IL); Timna NAFTALI, Tel-Aviv (IL)

(73) Assignees: The State of Israel, Ministry of Agriculture & Rural Development, Agricultural Research Organizat, Rishon-LeZion (IL); Mor Research Applications Ltd., Tel-Aviv (IL)

(21) Appl. No.: 16/085,623

(22) PCT Filed: Mar. 17, 2017
(86) PCT No.: PCT/IL2017/050338

§ 371 Date: Sep. 17, 2018

Related U.S. Application Data
(60) Provisional application No. 62/309,603, filed on Mar. 17, 2016.
(60) Provisional application No. 62/358,601, filed on Jul. 6, 2016.

(51) Int. Cl. C12N 5/071; G01N 33/50

(52) U.S. Cl. CPC C12N 5/0679; G01N 33/5008; C12N 2501/70; C12N 2500/76; C12N 2509/00; C12N 2533/90

(57) ABSTRACT

A method of preparing an ex-vivo intestinal culture model is provided. The method comprising:
(a) providing an intestinal tissue sample;
(b) treating the intestinal tissue sample with a protease cleaving an extracellular matrix (ECM) component so as to break cell-cell contacts and obtain an intestinal preparation which maintains the overall intestinal tissue structure including villi and/or intestinal tissue layer orientation;
(c) washing the preparation; and
(d) culturing the preparation.

### FIELD AND BACKGROUND OF THE INVENTION

The present invention, in some embodiments thereof, relates to an ex-vivo intestinal culture model, methods of producing same and uses thereof.

In contrast to cells of most other organs, normal human colon cells have been difficult to maintain in vitro. Currently available normal human intestinal epithelial cells are derived from the small intestine and exhibit undifferentiated features, while differentiated enterocytes remain in culture for only 10-12 days. Models of human intestine in culture are not suitable for studies of proliferation and differentiation. The cultured cells survive for only a few days. Co-culture of intestinal epithelial cells with fibroblasts or myofibroblasts could prolong survival. To improve survival, immortalization of colonic cells with genes from oncogenic viruses has been attempted. However, the transformed cells lost their typical epithelial morphology and did not polarize or differentiate.

There remains a need in the art for compositions and methods that provide a useful source of normal human intestinal epithelial cells which maintains in situ-like properties for use in studies of colon biology, screening for drug absorption and efficacy and for therapeutic uses, such as in transplantation or the treatment of colon lesions.

Various technologies are available to directly test the response of patient's cancer cells to a specific treatment, such as tumor derived cell lines generated from patients'tumors and patient-derived xenograft (PDX) models [see e.g. Crystal et al. (2014) Science 346, 1480-1486; Clevers et al. (2016) Cell 165, 1586-1597; and Hidalgo et al. (2014) Cancer Discov. 4, 998-1013]. However, these models are expensive and time-consuming and most importantly they do not retain the original tissue structure, which can have a marked effect on drug sensitivity.

Ex-vivo organ culture (EVOC) systems used in cancer biology are cultures of precision cut slices of the patient's tumor. In addition, EVOC has been used for diverse applications including the study of drug toxicity, viral uptake, susceptibility of tumors to radiation or specific anti-cancer drugs [see e.g. Vaira et al. (2010) Proc. Natl. Acad. Sci. U. S. A. 107, 8352-8356; Vickers et al. (2004) Chem. Biol. Interact. 150, 87-96; de Kanter et al. (2002) Curr. Drug Metab. 3, 39-59; Stoff-Khalili et al. (2005) Breast Cancer Res. BCR 7, R1141-1152; Merz et al. (2013) Neuro-Oncol. 15, 670-681; Gerlach et al. (2014) Br. J. Cancer 110, 479-488; Meijer et al. (2013) Br. J. Cancer 109, 2685-2695; Grosso et al. (2013) Cell Tissue Res. 352, 671-684; Vaira et al. (2010) PNAS 107, 8352-8356; Roife et al. (2016) Clin. Cancer Res. June 3, 1-10; Maund et al. (2014) Lab. Invest. 94, 208-221; Vickers et al. (2004) Toxicol Sci. 82(2):534-44; Zimmermann et al. (2009) Cytotechnology 61(3): 145-152); Parajuli et al. (2009) In Vitro Cell. Dev. Biol.—Animal 45:442-450; Koch et al. (2014) Cell Communication and Signaling 12:73; Graaf et al. Nature Protocols (2010) 1540-1551; Majumder et al. Nat. Commun. 6, 6169 (2015); US Patent Application Publication Nos: US2014/0228246, US2010/0203575 and US2014/0302491; and International Patent Application Publication No: WO2002/044344.

### Additional Related Art

US Patent Application Publication No. 20040175367

wwwdotmattek.com/products/epiintestinal/

### SUMMARY OF THE INVENTION

According to an aspect of some embodiments of the present invention there is provided a method of preparing an ex-vivo intestinal culture model, the method comprising:

(a) providing an intestinal tissue sample;

(b) treating the intestinal tissue sample with a protease cleaving an extracellular matrix (ECM) component so as to break cell-cell contacts and obtain an intestinal preparation which maintains the overall intestinal tissue structure including villi and/or intestinal tissue layer orientation;

(c) washing the preparation;

(d) culturing the preparation.

According to some embodiments of the invention, the protease is selected from the group consisting of collagenase, trypsin, papain, hyaluronidase, C. histolyticum neutral protease, thermolysin, elastase, BP protease dispase, and a combination of same.

According to some embodiments of the invention, the combination comprises collagenase and dispase.

According to some embodiments of the invention, the collagenase is collagenase 1A.

According to some embodiments of the invention, a concentration of dispase comprises 0.05 U/ml to about 0.15 U/ml units per 3 mm³ of the tissue.

According to some embodiments of the invention, a concentration of collagenase comprises 0.1 mg/ml to about 1.0 mg/ml per 3 mm³ of the tissue.

According to some embodiments of the invention, the washing is repeated at least 2-3 times.

According to some embodiments of the invention, the intestinal tissue sample is pathologic.

According to some embodiments of the invention, the intestinal tissue sample comprises an inflamed tissue.

According to some embodiments of the invention, the intestinal tissue is healthy.

According to some embodiments of the invention, the intestinal tissue is of a subject having an intestinal disease.

According to some embodiments of the invention, the intestinal tissue is of a subject having an inflammatory intestinal disease.

According to some embodiments of the invention, the inflammatory intestinal disease is selected from the group of Crohn's disease and ulcerative colitis.

According to some embodiments of the invention, the intestinal disease comprises cancer.

According to some embodiments of the invention, the treating the intestinal tissue sample with the protease cleaving an extracellular matrix (ECM) component so as to break cell-cell contacts comprises agitation of the intestinal tissue sample.

According to some embodiments of the invention, the culturing the preparation comprises placing the preparation in a culture vessel with villus surface facing up.

According to some embodiments of the invention, the culturing the preparation comprises placing the preparation on a tissue culture insert.

According to some embodiments of the invention, the culturing is for at least 2-4 days.

According to an aspect of some embodiments of the present invention there is provided an ex-vivo intestinal culture model obtainable according to the method as described herein.

According to an aspect of some embodiments of the present invention there is provided a method for screening a candidate agent for an effect on a mammalian tissue, the method comprising:

(a) contacting a candidate agent with the intestinal culture model as described herein, and

(b) determining the effect of the agent on the intestinal culture model.

According to an aspect of some embodiments of the present invention there is provided a method of selecting a drug for the treatment of an intestinal disease in a subject in need thereof, the method comprising:

(a) contacting the drug with the intestinal culture model as described herein of the subject; and

(b) determining the effect of the drug on the intestinal culture model, wherein sensitivity of the intestinal culture model to the drug indicates efficacy of the drug for the treatment of the intestinal disease.

According to an aspect of some embodiments of the present invention there is provided a method of treating an intestinal disease in a subject in need thereof, the method comprising:

(a) selecting a drug according to the method as described herein; and

(b) administering to the subject a therapeutically effective amount of a drug demonstrating efficacy for the treatment of the disease in the subject, thereby treating the disease in the subject.

According to some embodiments of the invention, the method further comprises stimulating an inflammatory response in the intestinal culture model prior to or concomitantly with the contacting.

According to some embodiments of the invention, the drug or agent is a drug or agent combination.

According to some embodiments of the invention, the drug comprises a cannabinoid.

According to some embodiments of the invention, the cannabinoid is synthetic.

According to some embodiments of the invention, the cannabinoid is extracted from cannabis.

According to some embodiments of the invention, the cannabinoid comprises THCA.

According to some embodiments of the invention, the determining the effect is by determining a secreted molecule.

According to some embodiments of the invention, the secreted molecule comprises a pro-inflammatory cytokine, an anti-inflammatory cytokine or a combination of same.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIGS. 1A-F are bar graphs showing secreted cytokines in biopsies. Production of TNF-α, IL-6 and IL-8 in biopsies treated with an ethanolic cannabis extract. Biopsies from healthy and sick zones of two patients were treated with an ethanolic cannabis extract. DX treatment served as positive control. Net values for the cannabis extract were obtained by subtracting the value obtained for the solvent control from that obtained for the extract. Supernatant for the evaluation of TNF-α level was taken one you after treatment while all the rest were taken after overnight incubation. (FIG. 1A) TNF-α production not standardized using extract dry weight. (FIG. 1B) TNF-α production standardized to extract dry weight. (FIG. 1C) IL-6 production not standardized using extract dry weight. (FIG. 1D) IL-6 production standardized using extract dry weight. (FIG. 1E) IL-8 production not standardized using extract dry weight. (FIG. 1F) IL-8 production standardized using extract dry weight. P1-H=Patient 1 Healthy zone; P1-S=Patient 1 sick zone; P2-H=Patient 2 Healthy zone; P2-S=Patient 2 sick zone; NT=non-treated; C2-F=Ethanolic cannabis extract produced from fresh flowers; DX=dexamethasone.

FIGS. 2A-D are bar graphs showing secretion of IL-10, IL-12, IL-17 and IL-27 as determined by ELISA. Levels of the different ILs were determined in biopsies treated with an ethanolic cannabis extract. Biopsies from healthy and sick zones of two patients were treated with an ethanolic cannabis extract. Non-treated biopsies from the same zones or biopsies treated with ethanol solution served as negative controls. DX treatment served as positive control. Net values for the cannabis extract were obtained by subtracting the value obtained for the solvent control from that obtained for the extract. Supernatant for ELISA assays were taken after overnight incubation. (FIG. 2A) IL-10 production not standardized using extract dry weight. (FIG. 2B) IL-12 production not standardized using extract dry weight. (FIG. 2C) IL-17 production not standardized using extract dry weight. (FIG. 2D) IL-27 production not standardized using extract dry weight. P1-H=Patient 1 Healthy zone; P1-S=Patient 1 sick zone; P2-H=Patient 2 Healthy zone; P2-S=Patient 2 sick zone; NT=non-treated; C2-F=Ethanolic cannabis extract produced from fresh flowers; DX=dexamethasone. Levels not connected by same letter are significantly different.

FIGS. 3A-D shows different images of stained biopsies with Risazurin (Alamar blue). Pictures were taken with a camera attached to the inverted microscope. (FIG. 3A) Patient 3—Healthy zone, non-treated. (FIG. 3B) Patient 3—Sick zone, Cannabis treated. (FIG. 3C) Patient 4—Healthy zone, non-treated. (FIG. 3D) Patient 4—Sick zone, ethanol treated.

FIG. 4 is a bar graph showing production of IL-6 in untreated vs. D/C (without D/C). Thus, treatment was with the cannabis extract and the control is the Non-Treated samples (NT). Differences in IL-6 levels when non-treated biopsies were treated with D/C vs. not treated with the D/C solution. Biopsies from the same patient (P5) were used for both treatments. These two groups of biopsies were then left untreated (NT) or treated with either a fresh (C2-FF) or dry (C2-DF) cannabis extract prepared from cannabis flowers using an ethanolic extraction protocol. Levels not connected by same letter are significantly different.

FIG. 5 is a bar graph showing that pre CBD has no anti-inflammatory activity. Production of IL-6 upon exposure to different cannabis treatments was measured in D/C treated biopsies after 18 hours. Biopsies from the same patient (P6) were used for all treatments a. NT=non-treated; C2-FF=ethanolic extraction from fresh flowers of C2 line; C2-DF=ethanolic extraction from dry flowers of C2; CBD=16.6 μM final concentration of pure CBD. Levels not connected by same letter are significantly different.

### DESCRIPTION OF SPECIFIC EMBODIMENTS OF THE INVENTION

The present invention, in some embodiments thereof, relates to an ex-vivo intestinal culture model, methods of producing same and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Development of an in vitro model system that is highly reflective of in vivo intestinal tissue would greatly facilitate the accurate determination of the effects of exogenous and endogenous agents on intestinal tissue and also the pathogenesis and prevention and treatment of intestinal diseases.

Whilst reducing embodiments of the invention to practice, the present inventors have devised a novel protocol for the treatment of intestinal tissue biopsies, which involves the use of a protease cleaving extracellular matrix (ECM) components, thereby breaking cell to cell contacts but maintaining the overall structure of the intestinal tissue (including villi and/or layer orientation). This procedure allows extensive washes which prevent tissue contamination. thereby increasing culture storage time. In addition, this procedure increases tissue sensitivity to external agents which is valuable for the use of these tissues in drug screening, personalized therapy, basic research and many more applications.

As is illustrated below and in the Examples section which follows, the present inventors were able to analyze the responsiveness of the intestinal culture model to cannabis extracts and pure cannabinoids (e.g., CBD) as well as to other plant extracts and dexamethasone, by analyzing pro-inflammatory/anti-inflammatory factors secretion. The responsiveness of the model, places it as an important tool in research and clinic.

Thus, according to an aspect of the invention there is provided a method of preparing an ex-vivo intestinal culture model, the method comprising:

(a) providing an intestinal tissue sample;

(b) treating said intestinal tissue sample with a protease cleaving an extracellular matrix (ECM) component so as to break cell-cell contacts and obtain an intestinal preparation which maintains the overall intestinal tissue structure (villi and/or tissue layer orientation);

(c) washing said preparation;

(d) culturing said preparation.

As used herein “intestinal” or “intestine” refers to the small intestine, large intestine, and/or rectum.

As used herein the term “tissue” refers to part of a solid organ (i.e., intestine) of an organism having some vascularization that includes more than one cell type and maintains at least some macro structure of the in-vivo tissue from which it was excised, in this case villus or villi.

As used herein “an intestinal tissue sample” refers to a tissue biopsy or an organoid.

As used herein an “organoid” refers to a simplified intestinal tissue structure generated from intestinal stem cells (wild type or carrying a mutation of an intestinal disease for instance), extracted from adult tissue and cultured in 3D media.

According to some embodiments, the tissue sample is a healthy tissue sample.

According to specific embodiments, the tissue is a mammalian tissue.

According to a specific embodiment, the tissue is a human tissue.

According to another specific embodiment, the tissue is a mouse or rat tissue.

According to some embodiments, the tissue sample is a pathologic tissue sample.

According to some embodiments, the tissue sample is a healthy tissue sample of a subject having an intestinal disease.

According to some embodiments, the tissue sample is a pathologic tissue sample of a subject having an intestinal disease.

According to some embodiment the tissue sample comprises a plurality of tissue samples (pathologic and/or healthy) from the same or different subjects (or organoids).

As used herein the term “pathological tissue” refers to a tissue causing a disease. Hence, elimination or treatment of such a tissue is expected to lead to at least partial alleviation and optimally cure as further defined hereinbelow. Specific examples of diseases amenable to treatment according to some embodiments of the present invention are described in details hereinbelow. According to specific embodiments the pathological tissue is an inflamed tissue, a fibrotic tissue or a cancerous tissue. According to a specific embodiment, the pathological tissue is an inflamed tissue.

According to specific embodiments the tissue is obtained surgically or by biopsy, laparoscopy, endoscopy, colonoscopy or as xenograft or any combinations thereof.

The tissue or the tissue slice of some embodiments of the present invention can be freshly isolated or stored e.g., cryopreserved (i.e. frozen) at e.g. liquid nitrogen.

According to specific embodiments, the tissue or the tissue slice is freshly isolated (i.e., not more than 24 hours after retrieval from the subject and not subjected to preservation processes).

According to specific embodiments, the tissue is cryopreserved following tissue retrieval and prior to cutting and/or proteolysis as described hereinbelow.

According to specific embodiments, the tissue is thawed prior to cutting and/or proteolysis.

According to specific embodiments, the tissue slice is cryopreserved following cutting or proteolysis.

According to specific embodiments, the tissue slice is thawed prior to culturing or proteolysis.

The size of the tissue can vary. According to some embodiments, the tissue surface area is 1-10 mm³. According to some embodiments, the tissue surface area is 1-8 mm³. According to some embodiments, the tissue surface area is 1-5 mm³. According to some embodiments, the tissue surface area is 2-5 mm³. According to some embodiments, the tissue surface area is 3 mm³. Every embodiment is considered a separate embodiment. For each embodiment the equivocal tissue weight should also be considered.

Cleavage of ECM components is the main process during ECM remodelling and is important for regulating ECM abundance, composition and structure, as well as for releasing biologically active molecules (such as growth factors). The ECM can be cleaved by different families of proteases. However, other proteases which naturally don't specifically cleave ECM are also contemplated herein and further discussed hereinbelow. Methods of assessing ECM degradation/remodelling are well known in the art and include but are not limited to histochemistry, immunohistochemistry and various protein assays such as Western blotting. Each of such methods can be used for selecting proteases that cleave an extracellular matrix component (i.e., polypeptide/peptide).

Matrix metalloproteinases (MMPs) are the main enzymes involved in ECM degradation. Their activity is low in normal conditions but increased during repair or remodelling processes and in diseased or inflamed tissue. MMPs are produced either as soluble or cell membrane-anchored proteinases and cleave ECM components with wide substrate specificities.

Adamalysins—This protein family includes ADAMs (a disintegrin and metalloproteinases) and ADAMTS (ADAMs with a thrombospondin motif). So far, 22 ADAM genes have been identified in humans but only 12 encode active proteinases. ADAMs are ‘sheddases’: they can cleave transmembrane protein ectodomains that are adjacent to the cell membrane, thus releasing the complete ectodomain of cytokines, growth factors, receptors and adhesion molecules. The disintegrin domains mediate cell-ECM interactions by binding integrins, and the Cys-rich domains interact with heparan sulphate proteoglycans (HSPGs). ADAM10, ADAM12 and ADAM15 can also cleave ECM proteins such as collagens.

In contrast to ADAMs, ADAMTS are secreted proteinases with thrombospondin type I-like repeats in their carboxy-terminal sequences. The aggrecanases (ADAMTS1, ADAMTS4, ADAMTS5, ADAMTS8, ADAMTS9, ADAMTS15 and ADAMTS20) are proteo-glycanolytic. ADAMTS2, ADAMTS3 and ADAMTS14 are pro-collagen N-propeptidases that process pro-collagens I, II and III and are important for depositing normal collagen fibrils onto the ECM in a tissue-specific manner. ADAMTS13, which cleaves von Willebrand factor, is involved in coagulation and thrombotic thrombocytopenic purpura (TTP).

Meprins—The meprins belong to the astacin family and are composed of two subunits (α and β) that are encoded by two different genes. The subunits can form heterocomplexes and homocomplexes linked by disulphide bridges. In contrast to meprin-α (which is a secreted protein as it loses its transmembrane domain by cleavage during biosynthesis), meprin-β is predominantly expressed on the cell surface but can be released from the membrane by shedding via ADAM10). Meprins can cleave ECM proteins such as collagen IV, nidogen and fibronectin. In addition, meprins may be necessary for the generation of mature collagen molecules by cleaving pro-collagen I that is assembled into collagen fibrils, which are important for skin tensile strength. Meprins can also indirectly regulate ECM remodelling by activating the other metalloproteinases. For example, ADAM10 is cleaved by meprin-β, and both meprin-α and meprin-β promote the cleavage of pro-MMP9 by MMP3, thus accelerating the activation of MMP9. Compared with the other metallo-proteinases, the roles of meprins in ECM remodelling are poorly understood.

Other enzymes important in ECM remodelling.

Serine (Ser) proteases can also target many ECM proteins. The two plasminogen activators urokinase and tissue plasminogen activator target plasminogen to generate plasmin, a protein that degrades fibrin, fibronectin and laminin. Moreover, the Ser protease elastase is released by neutrophils and promotes the breakdown of fibronectin and elastin, and the membrane-anchored Ser protease matriptase, which is expressed by epithelial cells, is important in maintaining the intestinal barrier.

In addition, cathepsins are found both extracellularly and intracellularly in lysosomes. Secreted cathepsins degrade extracellular ECM proteins, but many cells can also internalize ECM components such as collagen through endocytosis and degrade them in the lysosomes. Families of cathepsins include the Ser cathepsins (cathepsins A and G), Asp cathepsins (cathepsins D and E) and Cys cathepsins.

Heparanases or sulphatases can alter the properties of ECM PGs. Heparanase, an endoglucuronidase responsible for heparan sulphate (HS) cleavage, regulates the structure and function of HSPGs. This results in structural alterations of the ECM and the release of bioactive saccharide fragments and HS-bound growth factors and cytokines. Suphatase 1 and sulphatase 2 are secreted endosulphatases that remove 6-O-sulphate residues from HS and modulate HS binding to many cytokines and growth factors, including FGF1 and vascular endothelial growth factor (VEGF).

According to an embodiment of the invention, protease treatment can be a treatment using at least one member of proteases (enzymes) selected from collagenase (for any type of collagen), trypsin, papain, hyaluronidase, C. histolyticum neutral protease, thermolysin, elastase, BP protease and dispase, or a combination of two or more enzymes thereof.

As used herein “collagenase” refers to any enzyme that degrades a collagen and having an E.C. 3.4.24.7 classification.

As used herein “trypsin” refers to a serine protease from the PA clan superfamily, and having an EC 3.4.21.4 classification.

As used herein “dispase” refers to a protease which cleaves fibronectin, collagen IV, and to a lesser extent collagen I and having an E.C. 3.4.24.4 classification.

As used herein “papain”, also known as papaya proteinase I, is a cysteine protease (EC 3.4.22.2) enzyme present in papaya (Carica papaya) and mountain papaya (Vasconcellea cundinamarcensis). Papain belongs to a family of related proteins with a wide variety of activities, including endopeptidases, aminopeptidases, dipeptidyl peptidases and enzymes with both exo- and endo-peptidase activity. Members of the papain family are widespread, found in baculoviruses, eubacteria, yeast, and practically all protozoa, plants and mammals.

As used herein “hyaluronidase” refers to a family of enzymes that catalyse the degradation of hyaluronic acid (HA). According to their enzymatic mechanism, hyaluronidases are hyaluronoglucosidases (EC 3.2.1.35), i.e. they cleave the (1->4)-linkages between N-acetylglucosamine and glucuronate. The term hyaluronidase may also refer to hyaluronoglucuronidases (EC 3.2.1.36), which cleave (1->3)-linkages. In addition, bacterial hyaluronate lyases (EC 4.2.2.1) may also be referred to as hyaluronidases.

As used herein “C. histolyticum neutral protease” refers to neutral protease secreted by clostridium histolyticum.

As used herein “thermolysin” refers to EC 3.4.24.27, Bacillus thermoproteolyticus neutral proteinase, thermoase, thermoase Y10, TLN), a thermostable neutral metalloproteinase enzyme produced by the Gram-positive bacteria Bacillus thermoproteolyticus.

As used herein “elastase” refers to an enzyme that breaks down elastin, an elastic fibers that, together with collagen, determines the mechanical properties of connective tissue. The elastase has E.C. classifications as follows: 3.4.21.2, 3.4.21.36, 3.4.21.37, 3.4.21.65, 3.4.21.70, 3.4.21.71.

As used herein “BP protease” refers to a protease from Bacillus polymyxa.

According to an embodiment of the invention, the combination comprises collagenase and dispase.

The combination can be self made or purchased e.g., as a cocktail or in individual, separate packages.

According to an embodiment of the invention the collagenase is collagenase 1A.

According to an embodiment of the invention the concentration of dispase comprises 0.05-0.15 U/ml (e.g., 0.1 U/ml) per 3 mm³ of the tissue.

According to an embodiment of the invention the concentration of collagenase (assuming specific activity of 0.5-2 U/mg) comprises 0.1-1.0 mg/ml (e.g., 0.5 mg/ml) units per 3 mm³ of the tissue.

The conditions for such an protease treatment may be as follows: in an isotonic salt solution (e.g. PBS or Hanks' balanced salt solution) buffered at a physiologically acceptable pH (e.g. about pH 6 to 8, preferably about pH 7.2 to 7.6) at for example about 20 to 40° C., e.g., at about 25 to 39° C., for a time sufficient to degrade a connective tissue, for example, for several minutes (e.g., less than 10) to about several hours (e.g., to about 10 hours) e.g., about 1 to 180 minutes, e.g., 30 to 150 minutes, with a sufficient concentration for such degradation, for example, as described above.

The conditions for such an enzymatic treatment include, but not limited to, a treatment with a mixed enzyme containing collagenase. For example, the enzymatic treatment includes a treatment with a mixed protease solution comprising elastase and collagenase.

The protease can be purified or recombinantly expressed in suitable dedicated expression systems. It can be from a human source or animal, bacteria, insect or fungi source.

When using a protease combination, the enzymes may be used separately or alternatively in combination. In one embodiment, the intestinal tissue is digested using a mixture of collagenase and dispase.

The collagenase may be used at a concentration from about 0.1 mg/ml to about 1.0 mg/ml per 3 mm³ of tissue. The provided measures take into account specific activity of 0.5-2 U/mg. The values may change as the specific activity changes. According to other embodiments, the collagenase may be used at a concentration from about 0.1 mg/ml to about 0.9 mg/ml per 3 mm³ of tissue. According to other embodiments, the collagenase may be used at a concentration from about 0.1 mg/ml to about 0.8 mg/ml per 3 mm³ of tissue. According to other embodiments, the collagenase may be used at a concentration from about 0.1 mg/ml to about 0.7 mg/ml per 3 mm³ of tissue. According to other embodiments, the collagenase may be used at a concentration from about 0.1 mg/ml to about 0.6 mg/ml per 3 mm³ of tissue. According to other embodiments, the collagenase may be used at a concentration from about 0.2 mg/ml to about 0.6 mg/ml per 3 mm³ of tissue. According to other embodiments, the collagenase may be used at a concentration from about 0.3 mg/ml to about 0.6 mg/ml per 3 mm³ of tissue. According to other embodiments, the collagenase may be used at a concentration of about 0.5 mg/ml per 3 mm³ of tissue.

The dispase may be used at a concentration from about 0.05 U/ml to about 0.15 U/ml per 3 mm³ of tissue. According to other embodiments, the dispase may be used at a concentration from about 0.05 mg/ml to about 0.1 mg/ml per 3 mm³ of tissue. According to other embodiments, the dispase may be used at a concentration from about 0.05 mg/ml to about 0.09 mg/ml per 3 mm³ of tissue. According to other embodiments, the dispase may be used at a concentration from about 0.05 mg/ml to about 0.08 mg/ml per 3 mm³ of tissue. According to other embodiments, the dispase may be used at a concentration from about 0.05 mg/ml to about 0.1 mg/ml per 3 mm³ of tissue. According to other embodiments, the dispase may be used at a concentration from about 0.1 mg/ml to about 0.15 mg/ml per 3 mm³ of tissue. According to other embodiments, the dispase may be used at a concentration from about 0.05 mg/ml to about 0.12 mg/ml per 3 mm³ of tissue. According to other embodiments, the dispase may be used at a concentration from about 0.08 mg/ml to about 0.15 mg/ml per 3 mm³ of tissue. According to other embodiments, the dispase may be used at a concentration of about 0.1 mg/ml per 3 mm³ of tissue.

The tissue may be treated with the enzymes for about 5 minutes to about 5 hours. Alternatively the tissue may be treated with the enzymes for about 30 minutes to about 5 hours. Alternatively the tissue may be treated with the enzymes for about 30 minutes to about 4 hours. Alternatively the tissue may be treated with the enzymes for about 30 minutes to about 3 hours. Alternatively the tissue may be treated with the enzymes for about 30 minutes to about 4 hours. Alternatively the tissue may be treated with the enzymes for about 30 minutes to about 3 hour. In one embodiment, the tissue is treated with the enzymes for about 3 hours.

Treatment with the protease may comprise agitation to expose more parts of the tissue to the protease (orbital, vertical or any other) as long as the tissue maintains the overall structure e.g., villi and/or tissue layer orientation e.g., 50-70 rpm. Of note, measures are taken to maintain cell viability and prevent cell lysis, such that at least 90% of the cells remain intact and viable following proteolysis.

Following proteolysis, the intestinal preparation which maintains the overall intestinal tissue structure (e.g., villi and/or tissue layer orientation) is subjected to washing. The washing can be performed in volume excess of at least 2 fold. According to a specific embodiment, washing is effected at least one, twice or thrice, whereby each wash is followed by centrifugation of the preparation and discard of the supernatant which may comprise broken cells and debris. Washing is effected in a physiological buffer e.g., PBS or HBSS and other ingredients such as protease inhibitors included in the media beneath the inserts.

Following proteolysis the tissue can be cryopreserved or continue with further culturing.

For culturing, the tissue is sliced e.g., cut slices.

As used herein, the phrase “cut tissue slice” refers to a viable slice obtained from an isolated intestinal with reproducible, well defined thickness (e.g. ±5% variation in thickness between slices). Typically, the tissue slice is a mini-model of the tissue which contains the cells of the tissue in their natural with no selection of a particular cell type among the different cell type that constitutes the intestine. Cutting reduces sources of error due to variations in slice thickness and damage to cut surfaces, which both contribute to uneven gas and nutrient exchange throughout tissue slices; it enhances reproducibility; and allows adjacent slices to be evaluated for histology and compared pair-wise under different experimental conditions.

The slice section can be cut in different orientations (e.g. anterior-posterior, dorsal-ventral, or nasal-temporal) and thickness. According to a specific embodiment, the slice section(s) maintains all the tissue layers. The size/thickness of the tissue section is based on the tissue source and the method used for sectioning. According to specific embodiment the thickness of the cut slice allows maintaining tissue structure in culture.

According to specific embodiments the thickness of the cut slice allows full access of the inner cell layers to oxygen and nutrients, such that the inner cell layers are exposed to the sufficient oxygen and nutrients concentrations as the outer cell layers. This is determined by the tissue type but in general the tissue must be cut thick enough not to cause damage in handling but thin enough to allow diffusion of nutrients.

According to specific embodiments, the cut slice is between 50-1200 μm, between 100-1000 μm, between 100-500 μm, between 100-300 μm, or between 200-300 μm.

According to a specific embodiment, the cut slice is 200-300 μm.

Methods of obtaining tissue slices are known in the art and described for example in Roife et al. (2016) Clin. Cancer Res. June 3, 1-10; Vickers et al. (2004) Toxicol Sci. 82 (2):534-44; Zimmermann et al. (2009) Cytotechnology 61 (3): 145-152); Koch et al. (2014) Cell Communication and Signaling 12:73; and Graaf et al. Nature Protocols (2010) 5: 1540-1551, the contents of each of which are fully incorporated herein by reference. Such methods include, but are not limited to slicing using a vibratome, agarose embedding followed by sectioning by a microtome, or slicing using a matrix.

As a non-limiting example, the tissue is isolated treated with the protease as described herein and then placed in a physiological dissection media (e.g. HBSS-CONFIRM) which may be supplemented with antibiotics and protease inhibitors.

Following proteolysis, the tissue slice is placed on a tissue culture insert in a tissue culture vessel filled with culture medium. One slice or multiple slices can be placed on a single tissue culture insert (e.g., at least two pathologic, at least 2 healthy, at least one pathologic and at least one healthy). According to specific embodiments, one slice is placed on a single tissue culture insert.

According to specific embodiments, the culture vessel is filled with culture medium up to the bottom of the tissue slice (e.g. 4 ml of medium in a 6-well plate containing an insert).

The culture may be in a glass, plastic or metal vessel that can provide an aseptic environment for tissue culturing. According to specific embodiments, the culture vessel includes dishes, plates, flasks, bottles and vials. Culture vessels such as COSTAR®, NUNC® and FALCON® are commercially available from various manufacturers.

According to specific embodiments, the culture vessel is a tissue culture plate such as a 6-wells plate, 24-wells plate, 48-wells plate and 96-wells plate.

According to a specific embodiment, the culture vessel is a tissue culture 6-wells plate.

The culture medium used by the present invention can be a water-based medium which includes a combination of substances such as salts, nutrients, minerals, vitamins, amino acids, nucleic acids and/or proteins such as cytokines, growth factors and hormones, all of which are needed for cell proliferation and are capable of maintaining structure and viability of the tissue. For example, a culture medium can be a synthetic tissue culture medium such as DMEM/F12 (can be obtained from e.g. Biological Industries), M199 (can be obtained from e.g. Biological Industries), RPMI (can be obtained from e.g. Gibco-Invitrogen Corporation products), M199 (can be obtained from e.g. Sigma-Aldrich), Ko-DMEM (can be obtained from e.g. Gibco-Invitrogen Corporation products), supplemented with the necessary additives as is further described hereinunder. Preferably, all ingredients included in the culture medium of the present invention are substantially pure, with a tissue culture grade.

The skilled artisan would know to select the culture medium for intestinal culturing. The Examples section which follows provides an exemplary embodiment for culturing conditions.

According to specific embodiments of the invention, the culture medium comprises serum e.g. fetal calf serum (FCS, can be obtained e.g. from Gibco-Invitrogen Corporation products) or serum replacement.

According to specific embodiments, the culture medium is devoid of serum, or serum replacement.

According to a specific embodiment, the culture is xeno-free (devoid of animal constituents), e.g., animal cells, fluid or pathogens (e.g., viruses infecting animal cells), i.e., being xeno-free.

According to some embodiments of the invention, the culture medium can further include antibiotics (e.g., penicillin, streptomycin, gentamycin), anti-fungal agents (e.g. amphotericin B), L-glutamine or NEAA (non-essential amino acids) and optionally protease inhibitor(s).

According to a specific embodiment, the medium comprises serum and antibiotics.

According to a specific embodiment, the medium comprises DMEM/F12, 5% FCS, glutamine, penicillin, streptomycin, gentamycin and amphotericin B.

It should be noted that the culture medium may be periodically refreshed to maintain sufficient levels of supplements and to remove metabolic waste products that can damage the tissue. According to specific embodiments, the culture medium is refreshed every 12-72 hours, every 24-72 hours, every 24-48 hours or every 12-48 hours.

As used herein, the phrase “tissue culture insert” refers to a porous membrane suspended in a vessel for tissue culture and is compatible with subsequent ex-vivo culturing the preparation. The pore size is capable of supporting the preparation while it is permeable to the culture medium enabling the passage of nutrients and metabolic waste to and from the preparation, respectively. According to specific embodiments, the preparation is placed on the tissue culture insert, thereby allowing access of the culture medium to both the apical and basal surfaces of the tissue slice.

According to a specific embodiment, the culturing the preparation comprises placing the preparation in a culture vessel (on the insert) with villus surface facing up.

According to specific embodiments the pore size is 0.1 μm-20 μm, 0.1 μm-15 μm, 0.1 μm-10 μm, 0.1 μm-5 μm, 0.4 μm-20 μm, 0.4 μm-10 μm or 0.4 μm-5 μm.

According to specific embodiments the pore size is 0.4 mm-4 mm, 0.4 mm-1 mm, 1 mm-4 mm, 1 mm-3 mm or 1 mm, 2 mm.

According to specific embodiments, the tissue culture insert is sterile.

According to specific embodiments, the tissue culture inset is disposable.

According to specific embodiments, the cell culture insert is re-usable and autoclavable.

The cell culture insert may be synthetic or natural, it can be inorganic or polymeric e.g. alumina, Polytetrafluoroethylene (PTFE), titanium, Teflon, stainless steel, polycarbonate, nitrocellulose and cellulose esters. Cell culture inserts that can be used with specific embodiments of the invention are commercially available from e.g. Alabama R&D, Millipore Corporation, Costar, Corning Incorporated, Nunc, Vitron Inc. and SEFAR and include, but not limited to MA0036 Well plate Inserts, BIOCOAT™, Transwell®, Millicell®, Falcon®-Cyclopore, Nunc® Anapore, titanium-screen and Teflon-screen.

According to specific embodiments, the tissue culture insert is Millicell-HA or organotypic tissue culture inserts (Millipore).

According to specific embodiments, the culturing is for at least 2-4 days i.e., 48-96 hours.

According to specific embodiments, the culturing is effected in the presence of antibiotics but the concentration may be reduced as the treatment with the protease increases penetration of the antibiotics into the tissue thus preventing contamination.

Culturing can be effected in a humidified bioreactor or any other incubator useful for culturing.

Embodiments of the invention further relate to an ex-vivo intestinal culture model obtainable according to the method as described herein. Also provided herein is a collection of such culture models which can be stored in a bank, where each model is indexed according to various criteria mostly related to the donor (subject from which the tissue is derived) including gender, age, medical condition, intestinal disease, type of sample (e.g., small intestine, large intestine) and the like.

Also provided herein are methods of using the models as described herein.

Thus, according to some embodiments there is provided, a method for screening a candidate agent for an effect on a mammalian tissue, the method comprising:

(a) contacting a candidate agent with the intestinal culture model as described herein, and

(b) determining the effect of the agent on the intestinal culture model.

According to other embodiments there is provided a method of selecting a drug for the treatment of an intestinal disease in a subject in need thereof, the method comprising:

(a) contacting the drug with the intestinal culture model as described herein of the subject; and

(b) determining the effect of said drug on the intestinal culture model, wherein sensitivity of said intestinal culture model to said drug indicates efficacy of said drug for the treatment of said intestinal disease.

According to other embodiments there is provided a method of treating an intestinal disease in a subject in need thereof, the method comprising:

(a) selecting a drug according to the method as described herein; and

(b) administering to said subject a therapeutically effective amount of a drug demonstrating efficacy for the treatment of said disease in said subject, thereby treating the disease in the subject.

As used herein, the term “candidate agents” refers to oligonucleotides, polynucleotides, siRNA, shRNA genes, gene products, small molecules and pharmacological compounds (e.g., drugs) that is introduced in the intestinal culture model described herein to assay for their effect on the explants.

The term “contacting” refers to the placing candidate agents in the explant culture of mammalian intestinal cells.

Candidate agents (e.g., drugs) are screened for their effect on intestinal cells in the culture models of the invention. The effect of an agent or drug is determined by adding the agents to the intestinal culture models described above, usually in conjunction with a control culture model lacking the agent or drug. The growth of the intestinal tissue may be analyzed using methods well known in the art e.g., visually, cell viability, proliferation etc. The change in growth, differentiation, gene expression, proteome, phenotype with respect to markers, transport of agents, etc. in response to the agent or drug is measured and evaluated by comparison to control culture model. Agents of interest for analysis include any biologically active molecule with the capability of modulating, directly or indirectly, the growth rate of the culture model, for example genetic agents, monoclonal antibodies, protein factors, small molecule therapeutics, chemotherapeutics, radiation, anti-sense RNA, RNAi, and the like.

Candidate agents of interest are biologically active agents that encompass numerous chemical classes, organic molecules, which may include organometallic molecules, inorganic molecules, genetic sequences, etc. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, anti-inflammatory agents, hormones or hormone antagonists, ion channel modifiers, and neuroactive agents.

According to a specific embodiment, the candidate agent or drug is derived from cannabis.

According to a specific embodiment, the candidate agent or drug is a cannabinoid.

Phytocannabinoids or Cannabinoids are chemical compounds found in cannabis (marijuana), and other plants. Major Cannabinoids include, but are not limited to, Δ9-Tetrahydrocannabinol (THC), Cannibidiol (CBD), Cannabinol (CBN), Δ9-Tetrahydrocannabivarin (THCV), Cannabigerol (CBG), CBG, Cannabichromene (CBC), Cannabinoid Acids e.g., Δ9-Tetrahydrocannabinolic Acid (THCA), Cannabidiolic Acid (CBDA).

According to a specific embodiment, the cannabinoid is THCA (e.g., not including CBD for example).

Embodiments described herein relate to naturally occurring (purified, extracts), synthetics and analogs of agents derived from cannabis.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agent can also be polynucleotides and analogs thereof, which are tested in the screening assays of the invention by addition of the genetic agent to the intestinal culture model.

In some embodiments, the intestinal model culture is infected with an intestinal pathogen (bacterial or viral). Candidate agents are screened for anti-bacterial or anti-viral activity. Anti-bacterial or anti-viral activity of an agent can be assessed by monitoring growth, ultrastructure and viability of the explants. In other embodiments, the intestinal explant culture includes colon cancer cells, including cells suspected of being cancer stem cells.

To assess inflammation, the culture model may be stimulated with a pro-inflammatory agent prior to or concomitantly with the contacting.

According to a specific embodiment, the “pro-inflammatory agent” is a cytokine or interleukin.

A proinflammatory cytokine or an inflammatory cytokine is a type of cytokine (signaling molecule) that is excreted from immune cells and certain other cell types that promotes inflammation. Inflammatory cytokines are predominately produced by helper T cells (Th) and macrophages and involved in the upregulation of inflammatory reactions. Proinflammatory cytokines include interleukin-1 (IL-1), IL-12, and IL-18, tumor necrosis factor (TNF), interferon gamma (IFN-gamma), and granulocyte-macrophage colony stimulating factor.^{[3]}

In a certain application of the culture system, the intestinal culture model is used to assess whether certain agents cause intestinal toxicity. In these applications, the intestinal culture is exposed to the candidate agent or the vehicle and its growth and viability is assessed. In these applications, analysis of the ultrastructure of the intestinal explants is also useful.

In some embodiments, the intestinal model culture is infected with an intestinal pathogen (bacterial or viral). Candidate agents are screened for anti-bacterial or anti-viral activity. Anti-bacterial or anti-viral activity of an agent can be assessed by monitoring growth, ultrastructure and viability of the explants. In other embodiments, the intestinal explant culture includes colon cancer cells, including cells suspected of being cancer stem cells.

To assess inflammation, the culture model may be stimulated with a pro-inflammatory agent prior to or concomitantly with the contacting.

According to a specific embodiment, the “pro-inflammatory agent” is a cytokine or interleukin.

A pro-inflammatory cytokine or an inflammatory cytokine is a type of cytokine (signaling molecule) that is excreted from immune cells and certain other cell types that promotes inflammation. Inflammatory cytokines are predominately produced by helper T cells (Th) and macrophages and involved in the upregulation of inflammatory reactions. Proinflammatory cytokines include interleukin-1 (IL-1), IL-12, and IL-18, tumor necrosis factor (TNF), interferon gamma (IFN-gamma), and granulocyte-macrophage colony stimulating factor.

In a certain application of the culture system, the intestinal culture model is used to assess whether certain agents cause intestinal toxicity. In these applications, the intestinal culture is exposed to the candidate agent or the vehicle and its growth and viability is assessed. In these applications, analysis of the ultrastructure of the intestinal explants is also useful.

The agents/drugs are added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the lumen of the intestinal cysts and their effect compared to injection of controls.

Typically, agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations or combinations to obtain a differential response e.g., to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the growth rate.

According to a specific embodiment, the output or the measure of response to the agent/drug is by analyzing an inflammatory response, by determining a secreted molecule such as a pro-inflammatory or an anti-inflammatory cytokine.

Once a candidate agent drug is affirmed as beneficial is used for treating an intestinal disease, for example elevation in an anti-inflammatory response may indicate that the drug/agent is useful for treating an inflammation in a subject in need thereof.

As used herein “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The chief types of inflammatory bowel disease are Crohn's disease and ulcerative colitis (UC) and cancer.

In some embodiments, inflammatory bowel diseases fall into the class of autoimmune diseases, in which the body's own immune system attacks elements of the digestive system.

Other forms of IBD, which are not always classified as typical IBD include, but are not limited to:
Microscopic colitis subdivided into collagenous colitis and lymphocytic colitis
Diversion colitis
Behçet's disease
Indeterminate colitis

As mentioned, the treatment of cancer is also contemplated herein such as colon cancer, colorectal cancer,

The main types of small intestine cancer include adenocarcinomas, sarcoma Gastrointestinal stromal tumors. Carcinoid tumors and lymphomas (the latter being an immune system disease that may originate within the intestines).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Extraction of Cannabis

Fresh and baked (116 C for 1.5 h) flowers of C. sativa strain AD (C2F and C2B, respectively) were harvested from plants. They were either taken immediately for extraction and frozen at -80° C., or baked for 3 h at 150° C. prior to extraction. Fresh and baked Cannabis flowers (2 g) were pulverized with liquid nitrogen. Absolute ethanol was added to each tube containing the powder at a sample-to-absolute ethanol ratio of 1:4 (w/v). The tubes were mixed thoroughly on a shaker for 30 min and then the extract was filtered through a filter paper. The filtrate was transferred to new tubes. The solvent was evaporated with a vacuum evaporator. The dried extract was resuspended in 1 mL of absolute methanol and filtered through a 0.45-μm syringe filter. The filtered liquid was collected for the treatments, the resuspended extract was diluted for cell cultures and biopsies in enzyme-linked immunosorbent assay (ELISA) experiments. Sample dry weight was determined by crushing 1 g of plant material with known fresh weight and incubating overnight at 60° C., then weighing again for dry weight calculation.

### Chemical Characterization

### Standard Preparation

The cannabinoid standards cannabigerol (CBG), CBD, cannabidiolic acid (CBDA), cannabinol (CBN), cannabigerolic acid (CBGA), THC, cannabichromene (CBC) and THCA were diluted to 10 ppm concentration with methanol and then subjected to HPLC separation. For quantification of THC and THCA, the standards were dissolved in methanol at different concentrations from 5 ppm to 40 ppm.

### Sample Preparation

For HPLC, the dry extract (the ethanol crude) was resuspended in 1 mL methanol and filtered through a 0.45-μm syringe filter (Merck, Darmstadt, Germany). The filtered extract (the filtrate) was separated by HPLC into 9 fractions. For profile, the filtrate was diluted 50 times with methanol.

### HPLC Separation

Sample separation was carried out in an UltiMate 3000 HPLC system coupled with WPS-3000(T) Autosampler, HPG-3400 pump, and DAD-300 detector. The separation was performed on a Purospher RP-18 end capped column (250 mm×4.6 mm I.D.; Merck KGaA, Darmstadt, Germany) with a guard column (4 mm×4 mm I.D.). Solvent gradients were formed by isocratic proportion with 15% solvent A (0.1% acetic acid in water) and 85% solvent B (methanol) at a flow rate of 1.5 mL/min for 35 min. The compound peaks were detected at 220, 240 and 280 nm. The 220-nm peaks were taken for further processing. The extracts were fractionated into nine fractions according to the obtained chromatogram. F7 being the 7^{th} fraction which comprises THCA as the major cannabinoid.

### Determination of Dry Weight

1 gr of crushed material was wrapped in aluminum foil and the total weight was recorded. After overnight incubation at 60° C., the weight was determined again to calculate the dry weight.

### Culture of Biopsies

Biopsies (3 mm3) obtained from inflamed or healthy intestines and placed in PBS were treated with a Dispase (1 U/ml, Cat # 07923, StemCell Technologies Inc.) and Collagenase Type 1 (0.5 mg/ml with an activity of 0.5-2 U/mg Cat # 07902, StemCell Technologies Inc.) solution. Tubes were then incubated for 2-3h at 37° C.

After incubation, the tubes containing the biopsies were centrifuged at 5000 rpm for 1 minute. Then the supernatants were removed and tissues were washed three times with Hank's balance salt solution (HBSS). After each wash tubes were centrifuged as mentioned above. Then the tissues were placed on small Petri dish and cut into 2-3 pieces with a clean scalpel. Explants were cultured in groups of 2-3 with the villus surface facing up on microporous membranes (0.45-m pore size) contained in either Millicell-HA or organotypic tissue culture inserts (Millipore) placed inside 6-well plastic tissue culture dishes (Costar 3506) along with 1.5 ml of tissue culture medium. The tissue culture medium was Dulbecco's modified Eagle's medium supplemented with 10% heat-inactivated fetal calf serum, penicillin (100units/ml), streptomycin (100 μg/ml), leupeptin (50 μg/ml), PMSF (1 mM), soybean trypsin inhibitor (50 μg/ml). In cases dexamethasone (DX) was included in the media, its concentration was 200 μg/ml. This is followed by treating the samples with Control and extracts treatments (cannabis or other). In cases TNF-α is added to induce ILs expression the concentration was 50 μg/ml. Cultures are then incubated at 37° C. and gassed hourly with 95% oxygen and 5% carbon dioxide. For evaluation of TNF-α expression, supernatants are taken after 1 h of incubation. For other ILs, supernatants are taken after overnight incubation unless mentioned differently. The supernatants from biopsies are used for determination of cytokine profile by measuring their levels using commercial ELISA Kits. The following cytokines were measured: TNF Alfa (the supernatant was taken after 1-3 h), for the rest of the cytokines and interleukins supernatant was analyzed after 16 hours. The following factors were analyzed: IFN gamma, TFG beta, IL-10, IL-23/IL-23R, IL-12, IL-1 (IL 113), IL 2, IL-8/IL-8R, IL-6, IL-17, IL-13, IL-5, IL-4, IL-3. Levels of secreted factors from inflamed and healthy tissue and from cannabis treated and not treated tissue were compared. After all samples are taken, tissue viability will be assessed by addition of Alamar blue (Risazurin) as described below.

### Viability Test Using Alamar Blue (Risazurin)

The assay was done according to manufacturer's instructions (R&D Systems Cat # AR002). In biopsies experiments, if the remaining supernatant (after removing supernatant for ELISA experiments) was less than 1 ml, additional media was added to obtain a total volume of 1.5 ml. Then 10% v/v of reagent was added to each well. Plates were then covered with aluminum foil and plates were re-incubated for 4 h at 37° C. and gassed hourly with 95% oxygen and 5% carbon dioxide. Change of the media color from purple to pink indicated viable tissue. Fluorescence reads were obtained using 544 nm excitation and 590 nm emission wavelengths. However, in biopsies experiments, since the color change cannot always be seen in the media, observation under an inverted microscope is recommended and then pink color can be seen within the tissues.

### EXAMPLE 1

### Evaluation of 7 Cytokines in Healthy and Sick Biopsies

Biopsies obtained from both healthy and sick intestinal samples of two patients were cultured overnight incubation using the full protocol (D/C) described in material and methods section hereinabove. Seven different cytokines were evaluated following overnight incubation using specific ELISA kits manufactured by R&D Systems.

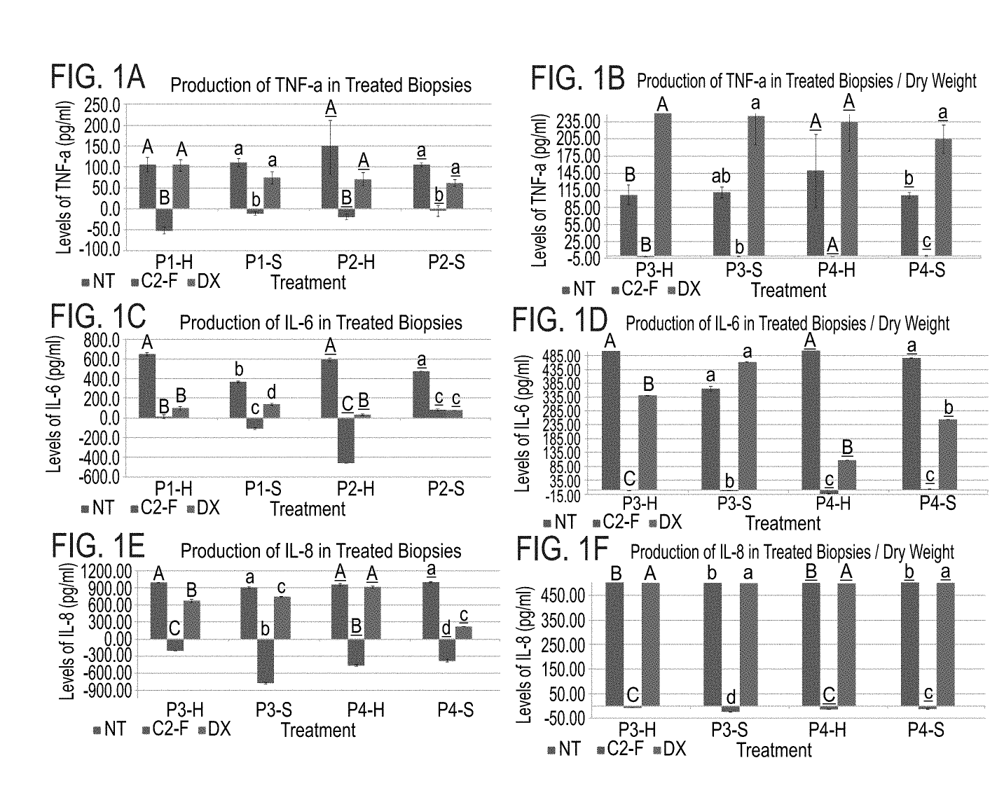

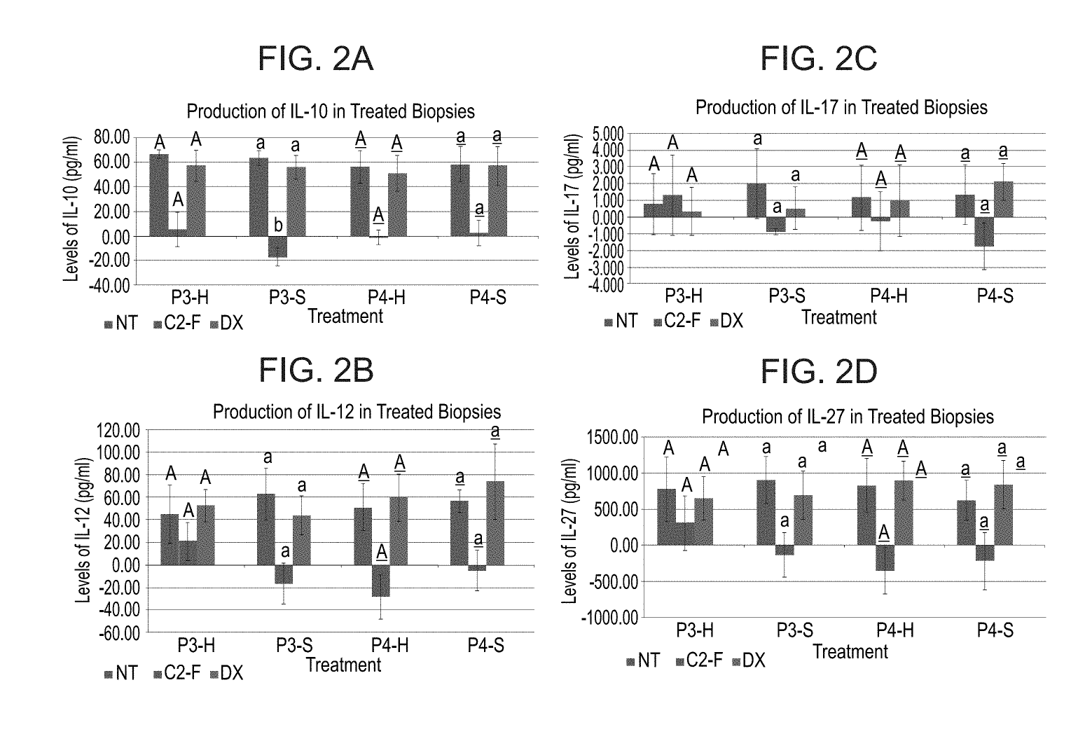

The cytokines tested included TNF-α, IL-6, IL-8, IL-10, IL-12, IL-17 and IL-27. In addition to the ethanolic cannabis extract was tested for its ability to reduce inflammation. After supernatant sampling, additional media was added to the wells and Resazurin (alamar blue) viability assay was carried out. The ELISA results clearly showed two groups of ILs. Responsive cytokines that show significant levels of expression (FIGS. 1A-F) on one hand vs. other ILs in which basal level were obtained which didn't allow a clear conclusion (FIGS. 2A-D).

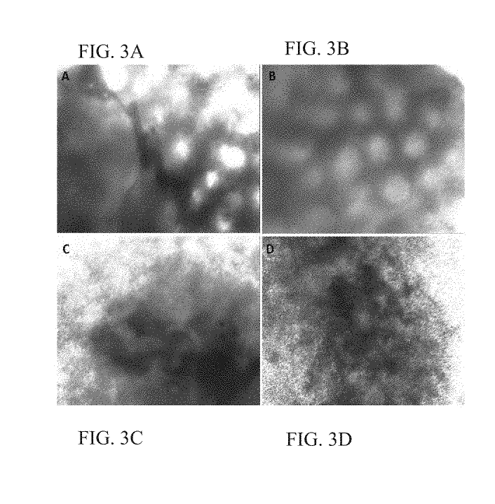

Cannabis C2F extract treatment results in reduction of all TNF-α, IL-6 and IL-8. As expected from its transient and fast expression it seems measuring TNF-α levels after one hour of treatment results in better results compared to measurement after overnight treatment (FIGS. 1A and B). In all three cytokines (TNF-α, IL-6 and IL-8) it seems that personal differences are more noticeable when levels of expression are not standardized using dry weight (FIGS. 1A, C and E). However, some differences, such as different reaction to steroids (DX) can be detected when standardizing to dry weight (FIGS. 1B and D). Viability of cells was observed only within the tissues and not in the media (FIGS. 3A-D) implying that cell are still viable.

Several more experiments were done to corroborate the above results. These are summarized in Table 1 below(C2F and F7 and dexamethasone).

TABLE 1
Further results of IL-8 secretion are shown below:

| | | C2F | | F-7 | | Dexamethasone | |
|---|---|---|---|---|---|---|---|
| | | | % Reduction | | % Reduction | | % Reduction |
| # Patient | NT | Value | from NT | Value | from NT | Value | from NT |
| PS-3-S | 900.1 | 34.2 | | | | 742.6 | 17.5 |
| PS-3-H | 991.6 | 656.7 | 33.8 | | | 670.1 | 32.4 |
| PS-4-S | 998.6 | 382.1 | 61.7 | | | 216.9 | 78.3 |
| PS-4-H | 956.8 | 424.6 | 55.6 | | | 915.4 | 4.3 |
| PH-5-H | 355.5 | 70.7 | 80.1 | | | | |
| PH-6-H | 389.2 | 11.2 | 97.1 | | | | |
| PS-7-S | 752.2 | 22.1 | 97.1 | 1.6 | 99.8 | 793.9 | −5.6 |
| PS-7-H | 873.4 | 27.3 | 96.9 | 78.2 | 91.0 | 745.1 | 14.7 |
| PS-8-H | 704.8 | | | | | 215.3 | 69.5 |
| PS-11-S | 444.0 | −2.7 | 100.6 | | | 74.2 | 83.3 |
| PS-11-H | 666.2 | −4.2 | 100.6 | | | 34.9 | 94.8 |
| PS-12-S | 852.3 | 82.3 | 90.3 | | | 817.8 | 4.0 |
| PS-12-H | 941.7 | 70.5 | 92.5 | | | 530.6 | 43.7 |
| PH-13-H | 835.5 | 32.6 | 96.1 | | | 844.6 | −1.1 |
| PS-14-S | 878.7 | 36.2 | 95.9 | | | 666.8 | 24.1 |
| PS-14-H | 870.9 | 15.8 | 98.2 | | | 485.8 | 44.2 |
| PH-15-H | 843.4 | 61.0 | 92.8 | | | 451.9 | 46.4 |
| PS-16-S | 589.6 | 36.4 | 93.8 | | | 96.7 | 83.6 |
| PS-16-H | 269.9 | 21.9 | 91.9 | | | 166.5 | 38.3 |
| PS-17-S | 1069.3 | 102.8 | 90.4 | | | 1064.0 | 0.5 |
| PS-17-H | 976.1 | 58.9 | 94.0 | | | 303.5 | 68.9 |
| PS-18-S | 759.2 | 88.4 | 88.4 | | | 484.9 | 36.1 |
| PS-18-H | 986.0 | 22.5 | 97.7 | | | 331.0 | 66.4 |
| PS-19-S | 225.0 | −1.9 | 100.8 | 3.8 | 98.3 | 80.5 | 64.2 |
| PS-19-H | 127.7 | 1.1 | 99.2 | −1.9 | 101.5 | 86.0 | 32.6 |
| PS-20-S | 302.7 | 9.7 | 96.8 | 9.0 | 97.0 | | |
| PS-20-H | 356.2 | 10.6 | 97.0 | 4.7 | 98.7 | | |
| PS-21-S | 820.365 | 17.8 | 97.8 | 19.65 | 97.6 | | |
| PS-21-H | 641.615 | 3.24 | 99.5 | 3.26 | 99.5 | | |
| PS-22-S | 667.16 | 201.1 | 69.86 | 79.70 | 88.1 | | |

P—patient;
1^{st} S/H sick or healthy respectively;
2^{nd} S/H sick or healthy tissue within the patient respectively;

### EXAMPLE 2

### Is the Dispase/Collagenase Treatment Effective?; Pure CBD (Cannabidiol) Has No Anti-Inflammatory Effect

The following experiment was performed on biopsies from two healthy patients and had two main goals:

(1) To evaluate whether the treatment of biopsies with dispase/collagenase (D/C treatment) solution increases the levels of IL-6 detected.

(2) To determine whether application of pure CBD reduces IL-6 levels, i.e. exhibits an anti-inflammatory activity. Treatment was performed with purified CBD (Restek, Pa., USA) at 25 μM, was done as described above, only CBD was used instead of C2F, C2B or F7.

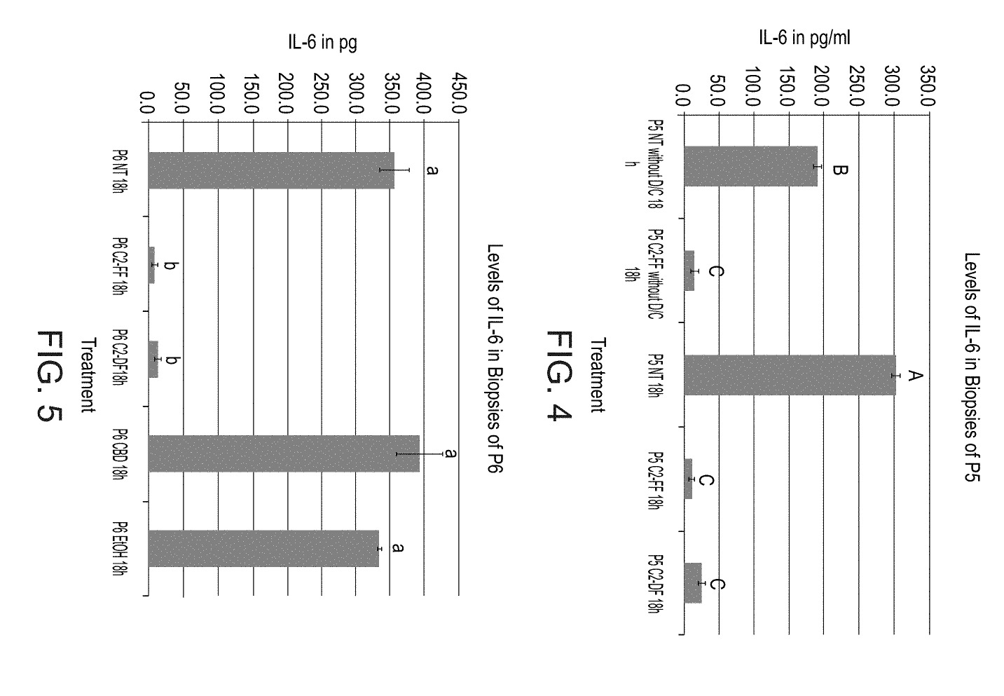

In order to test the effectiveness of the D/C treatment, biopsies from a healthy patient (P5) were used. Five different treatments were carried out. On one hand biopsy pieces that were not D/C treated were left untreated or were treated with an ethanolic cannabis extract prepared from fresh flowers (C2-FF). On the other hand additional D/C treated biopsy pieces were either left untreated or exposed to the same cannabis fresh flower extract or a dry cannabis flower extract prepared using the same conditions. IL-6 levels were measured after 18 hours of incubation, using a R&D Systems' ELISA Kit (FIG. 4).

Clear conclusions can be drawn from both experiments described above. Although IL-6 levels can be detected in D/C untreated biopsies, such treatment enables detection of higher levels of this cytokine, rendering the assay far more sensitive. Comparison of the IL-6 detected levels in D/C treated biopsies vs. untreated ones reveals an approximately twofold level respectively. It is suggested that the main reason for such a difference is caused by the higher levels of IL-6 that solubilized in the media when biopsies are D/C treated. Regardless of the D/C treatment, a following treatment with a cannabis extract from fresh or dry flowers results in a strong reduction of IL-6 levels.

The second experiment performed clearly demonstrates that different extracts or preparations of cannabinoids may affect intestinal cells in different ways. These results revealed that pure CDB doesn't harbor any anti-inflammatory activity (FIG. 5). This might be explained by removal of cannabis-derived substances in the purification process of CBD or by additive or suppressive molecules in different cannabis extraction/fractions. The differences observed between pure CBD and fresh or dry cannabis extracts point out again the basis for personal treatment using different extracts/fractions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of preparing an ex-vivo intestinal culture model, the method comprising:
(a) providing an intestinal tissue sample;
(b) treating said intestinal tissue sample with a protease cleaving an extracellular matrix (ECM) component so as to break cell-cell contacts and obtain an intestinal preparation which maintains the overall intestinal tissue structure including villi and/or intestinal tissue layer orientation;
(c) washing said preparation; and
(d) culturing said preparation.

2. (canceled).

3. The method of claim 1, wherein said protease comprises a protease combination comprising collagenase and dispase.

4. The method of claim 3, wherein said collagenase is collagenase 1A.

5. The method of claim 3, wherein a concentration of dispase comprises 0.05 U/ml to about 0.15 U/ml units per 3 mm³ of the tissue.

6. The method of claim 3, wherein a concentration of collagenase comprises 0.1 mg/ml to about 1.0 mg/ml per 3 mm³ of the tissue.

7. The method of claim 1, wherein said washing is repeated at least 2-3 times.

8. The method of claim 1, wherein said intestinal tissue sample is pathologic.

9. The method of claim 7, wherein said intestinal tissue sample comprises an inflamed tissue.

10-15. (canceled)

16. The method of claim 1, wherein said culturing said preparation comprises placing said preparation in a culture vessel with villus surface facing up.

17. The method of claim 1, wherein said culturing said preparation comprises placing said preparation on a tissue culture insert.

18. The method of claim 1, wherein said culturing is for at least 2-4 days.

19. An ex-vivo intestinal culture model obtainable according to the method of claim 1.

20. A method for screening a candidate agent for an effect on a mammalian tissue, the method comprising:
(a) contacting a candidate agent with the intestinal culture model of claim 19, and
(b) determining the effect of the agent on the intestinal culture model.

21. A method of selecting a drug for the treatment of an intestinal disease in a subject in need thereof, the method comprising:
(a) contacting the drug with the intestinal culture model of claim 19 of the subject; and
(b) determining the effect of said drug on the intestinal culture model, wherein sensitivity of said intestinal culture model to said drug indicates efficacy of said drug for the treatment of said intestinal disease.

22. A method of treating an intestinal disease in a subject in need thereof, the method comprising:
(a) selecting a drug according to the method of claim 21; and
(b) administering to said subject a therapeutically effective amount of a drug demonstrating efficacy for the treatment of said disease in said subject, thereby treating the disease in the subject.

23. The method of claim 20, further comprising stimulating an inflammatory response in said the intestinal culture model prior to or concomitantly with said contacting.

24. The method of claim 20, wherein said drug or agent is a drug or agent combination.

25. The method of claim 20, wherein said drug comprises a cannabinoid.

26-28. (canceled)

29. The method of claim 20, wherein said determining said effect is by determining a secreted molecule.

30. (canceled)
